(19) United States
(12) Patent Application Publication
(10) Pub. No.: US 2008/0195137 A1
(43) Pub. Date: Aug. 14, 2008

(54) Devices and Methods for Aneurysm Treatment

(76) Inventors: Cargill H. Alleyne, Martinez, GA (US); Jeffrey A. Stone, Martinez, GA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/666,272

(22) PCT Filed: Oct. 25, 2005
(86) PCT No.: PCT/US2005/038984

§ 371 Date: Mar. 19, 2008

Related U.S. Application Data
(60) Provisional application No. 60/651,496, filed on Oct. 26, 2004.

(51) Int. Cl. A61M 29/00; A61F 2/84

(52) U.S. Cl. 606/195; 606/194; 623/1.11

(57) ABSTRACT

A device for treating a fusiform aneurysm includes an elongate catheter and an expandable member detachably coupled with the elongate catheter at or near its distal end and adapted to assume a fusiform expanded shape. In its expanded shape the external surface of the expandable member will contact an inner wall of the aneurysm, thus preventing blood from passing between the external surface of the expandable member and the inner wall of the aneurysm. The expandable member includes an inflation lumen for expansion and a blood flow lumen to allow blood to flow from a portion of a blood vessel proximal to the aneurysm, through the blood flow lumen, to a portion of the blood vessel distal to the aneurysm. The expandable member is adapted to retain an expanded configuration and remain in place within the aneurysm when detached from the elongate catheter body.

### CROSS-REFERENCES TO RELATED APPLICATIONS

This application is a continuation of U.S. Provisional Patent Application 60/651,496, filed Oct. 26, 2004, the contents of which are hereby incorporated by reference.

### BACKGROUND OF THE INVENTION

The present application relates to medical devices and methods. More specifically, the invention relates to devices and methods for treating aneurysms.

An aneurysm is a sac formed by localized dilatation of the wall of an artery, a vein, or the heart. Aneurysms occur in many different blood vessels throughout the body, with two of the more common sites including intracranial blood vessels and the abdominal aorta. Typically, aneurysms form when a localized weakness in a vessel wall causes the vessel to locally expand with blood pressure, creating the aneurismal sac. Blood pressure then increases within the sac, due to fluid flow dynamics, causing the sac to grow larger. As the aneurysm grows, the blood vessel wall becomes continually thinner and weaker, and rupture of the vessel becomes increasingly serious risk. Ruptured aneurysms are often catastrophic. Intracranial aneurysm rupture, for example, frequently causes stroke and may even lead rapidly to death. Abdominal aortic aneurysm (AAA) rupture is also associated with very high rates of morbidity and mortality.

A variety of surgical and non-surgical procedures have been developed to treat various types of aneurysms. For example, an open surgical procedure is traditionally used to access and place a graft across an AAA. More recently, intravascular, catheter-based devices and methods have been developed for placing grafts across AAAs, although many such techniques and devices have met with limited success. Intravascular techniques are also commonly used to treat intracranial aneurysms. One such method, for example, involves placing a metallic coil or other substance in the aneurismal sac to take up space and thus prevent blood flow through the sac, thus minimizing further growth of the aneurysm. Various methods and devices for treating aneurysms are described, for example, in U.S. Pat. Nos. 6,511,468; 6,395,019; 6,350,270; 6,331,191; 5,843,160; 5,785,679; 5,665,117; 5,534,024; 5,330,528; 5,156,620 and 5,041,090, and in U.S. Patent Application Publication Nos.: 2003/0225453; 2003/0216802; 2003/0028209 and 2002/0165572.

One ongoing challenge has been the treatment of fusiform aneurysms and other large aneurysms in the cerebral vasculature. Fusiform aneurysms are round and taper from the middle toward each end. Additionally, fusiform aneurysms are typically larger than other intracranial aneurysms. The shape and size of such aneurysms makes them difficult, and sometimes impossible, to treat with currently available intravascular techniques. Placement of metal coils or other substances into the aneurysm does not work, because the sac circles the vessel, rather than being an asymmetrical outpouching of the vessel to one side. Attempts have been made to place stents across fusiform aneurysms, but stents by themselves typically do not stay in place across a large, fusiform aneurysm. Furthermore, the irregular blood flow patterns present in the aneurysm tend to twist and bend the vessel and thus twist and bend the stent, which may cause it to break or dislodge. Due to the inadequacies of coil and stent placement in such aneurysms, common treatments include either ligating or occluding the vessel feeding the aneurysm and, optionally, bypassing blood flow around it, or clip reconstruction of the vessel. Such procedures, however, typically involve invasive, open surgical procedures and thus pose additional risks not typically involved in less invasive, intravascular procedures. Therefore, no ideal treatment solution has yet been described for fusiform intracranial aneurysms. Similar challenges also exist for fusiform aneurysms in other locations in the body.

Therefore, a need exists for devices and methods for treating fusiform aneurysms. Ideally, such devices and methods would be minimally or at least less invasive, relative to surgical techniques. For example, it would be ideal to have catheter-based, intravascular treatment devices and methods for fusiform aneurysms. Ideally, such devices and methods would be suited for treatment of intracranial fusiform aneurysms as well as other fusiform aneurysms at different locations throughout the body. At least some of these objectives will be met by the present invention.

### BRIEF SUMMARY OF THE INVENTION

In one aspect of the present invention, a device for treating a fusiform aneurysm includes an elongate catheter having a proximal end and a distal end and an expandable member detachably coupled with the elongate catheter at or near the distal end. The expandable member is adapted to assume a fusiform expanded shape such that its external surface will contact an inner wall of the aneurysm, thus preventing blood from passing between the external surface of the expandable member and the inner wall of the aneurysm. The expandable member also includes an inflation lumen, to allow expansion of the expandable member, and a blood flow lumen. The blood flow lumen extends through the expandable member to allow blood to flow from a portion of a blood vessel proximal to the aneurysm, through the blood flow lumen, to a portion of the blood vessel distal to the aneurysm. The expandable member retains an expanded configuration and remains in place within the aneurysm when detached from the elongate catheter body. Thus, when deployed within a blood vessel, the expandable member prevents blood from flowing through the aneurismal sac while maintaining blood flow through the vessel via the blood flow lumen.

In some embodiments, the fusiform aneurysm being treated comprises a large, intracranial aneurysm. In other embodiments, other aneurysms, such as an AAA, may be treated. To help affix or adhere the external surface of the expandable member to the inner wall of the blood vessel, the device may optionally include one or more substances coupled with the expandable member's external surface. Such substances may include, but are not limited to, collagen, tissue adhesives or a polymeric matrix. The expandable member itself may have any of a number of different shapes and sizes, in various embodiments. In fact, in one embodiment, each expandable member may be custom made, based on a three-dimensional model made of the aneurysm to be treated. Although a number of sizes are possible, in some embodiments, expandable members used for treating intracranial aneurysms may have a length of between about 10 mm and 40 mm, an outer diameter of between about 6 mm and 30 mm, and a blood flow lumen diameter of between about 3 mm and 6 mm.

Optionally, the device may further include at least one balloon-expandable or self-expanding stent adapted for balloon expansion within the blood flow lumen of the expandable member to support the blood flow lumen. In some embodiments, the catheter may be adapted to deploy such stents within the blood flow lumen. A stent deployed within the blood flow lumen of the expandable member generally provides added support and strength for the expandable member, thus helping to prevent twisting, bending or other movement of the device within the vessel. In one embodiment, the stent extends through the blood flow lumen, with its ends in contact with the blood vessel wall on either side of the expandable member. Thus, the stent may act as an anchor to help hold the expandable member in place, as well as adding support to the expandable member.

A number of different techniques may be used for deploying the expandable member via the catheter. In one embodiment, for example, the expandable member further includes a self-sealing, one-way valve for detachably coupling the expandable member to the catheter. The one-way valve allows an inflation medium, such as saline, to be introduced into the expandable member and prevents it from escaping back through the valve. Detaching the catheter from the expandable member seals the valve, thus sealing the inflation medium within the expandable member. In some embodiments, the valve detachably couples the expandable member to the catheter electrolytically, while in alternative embodiments the valve may coupled the expandable member to the catheter hydrostatically or by other means.

In some embodiments, the expandable member further comprises at least one anchoring member coupled with its outer surface. The anchoring member(s) are adapted to anchor the expandable member to the inner wall of the aneurysm. Optionally, the expandable member may further comprise at least one radiopaque material or marker for facilitating visualization of the device.

In another aspect of the present invention, a method for treating a fusiform aneurysm involves expanding an expandable member into a fusiform shape within the aneurysm to cause an external surface of the expandable member to contact an inner wall of the aneurysm to prevent blood from passing between the external surface of the expandable member and the inner wall of the aneurysm. The expanded expandable member is then left within the aneurysm to prevent blood from passing between the external surface of the expandable member and the inner wall of the aneurysm. At the same time, the expandable member allows blood to flow through the aneurysm via a blood flow lumen. The expandable member includes an inflation lumen to allow expansion of the expandable member and a blood flow lumen extending through the expandable member to allow blood to flow from a portion of a blood vessel proximal to the aneurysm, through the blood flow lumen, to a portion of the blood vessel distal to the aneurysm.

Typically, though not necessarily, the expandable member is expanded via an elongate catheter device detachably coupled with the expandable member and in fluid communication with the inflation lumen, in which case the step of leaving the expandable member within the aneurysm involves detaching the catheter from the expandable member. In some embodiments, the expandable member is inflated with saline, though other suitable inflation media may be used in various alternative embodiments. In some embodiments, detaching the catheter from the expandable member automatically seals a one-way inflation valve on the expandable member.

Optionally, the method may also involve placing at least one stent within the blood flow lumen to support the blood flow lumen. The stent may be placed either by balloon expansion or by placing a self-expanding stent, in various embodiments. In some embodiments, the expandable member is expanded and the stent is placed using one catheter device. The method may also optionally include performing an angiogram of the aneurysm during the expanding step to determine whether blood is flowing between the external surface of the expandable member and the inner wall of the aneurysm. In some embodiments, an additional angiogram of the aneurysm may be obtained after the expanding step to confirm the absence of blood between the external surface of the expandable member and the inner wall of the aneurysm.

In another aspect of the present invention, a method for making an expandable device to treat a fusiform aneurysm involves acquiring at least one image of the fusiform aneurysm, preparing at least one three-dimensional model of the fusiform aneurysm, based on the at least one image, and forming the expandable device to have a shape approximating that of the three-dimensional model. By using this method, custom-made expandable members may be made to fit aneurysms having different sizes and shapes. In some embodiments, the model comprises a mold, and forming the expandable device comprises disposing an elastomeric material over the mold. As discussed above, in some embodiments, the expandable device comprises an inflatable balloon having at least one self-sealing, one-way valve. In alternative embodiments, a number of differently sized and shaped expandable members may be provided to a user, without custom-fitting each expandable member, and a size and shape may be chosen in each case based on the size and shape of the aneurysm being treated.

These and other aspects and embodiments of the invention will be described in further detail below in reference to the attached drawing figures.

### BRIEF DESCRIPTION OF THE DRAWINGS

FIG. 1 is a cross-sectional view of a portion of an intracranial blood vessel with a fusiform aneurysm, illustrating in side-view a device for treating the aneurysm, according to one embodiment of the present invention.

FIG. 2A is a cross-sectional view of an expandable device for treating a fusiform aneurysm, according to one embodiment of the present invention.

FIG. 2B is a perspective view of the expandable device shown in FIG. 2A.

FIG. 2C is an end-on view of the expandable device shown in FIGS. 2A and 2B.

FIGS. 3A-3C illustrate a method for placing a device for treating a fusiform aneurysm, according to one embodiment of the present invention.

### DETAILED DESCRIPTION OF THE INVENTION

Devices and methods of the present invention generally provide for treatment of fusiform aneurysms, especially but not exclusively intracranial fusiform aneurysms. Devices include an expandable member having an inflation lumen and a blood flow lumen. When expanded, the expandable member is configured to prevent blood flow between the outer surface of the expandable member and the inner surface of the aneurysm, while allowing blood flow through the blood flow lumen. Thus, the expandable member fills the space of the aneurysm while also maintaining blood flow through the treated vessel. The expandable member is placed in the aneurysm and expanded via an intravascular, catheter-based technique.

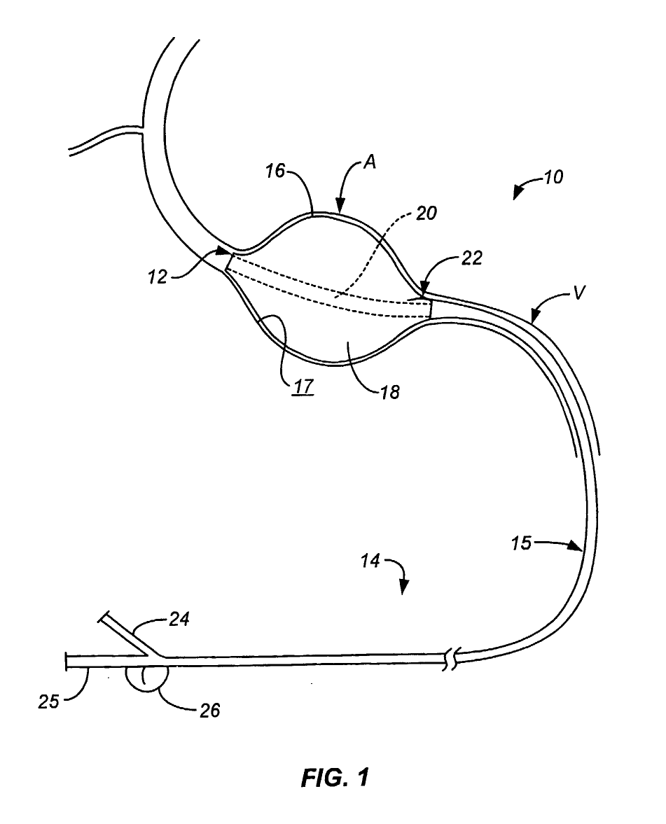

Referring now to FIG. 1, a fusiform aneurysm treatment device 10 is shown in place within a vessel V that has a fusiform aneurysm A. Treatment device 10 generally includes an expandable member 12 and an elongate delivery catheter 14 for delivering and expanding expandable member 12. Expandable member 12 includes an inflation lumen 18, a self-sealing one-way valve 22 opening into inflation lumen 18, a blood flow lumen 20, and an outer surface 16. Expandable member 12 is generally configured such that when it is expanded, outer surface 16 contacts and lies flush with an inner surface 17 of the aneurysm A. For clarity, a small space is shown between outer surface 16 and inner surface 17, as if expandable member 12 is not fully expanded. In practice, expandable member 12 will be expanded until it contacts the aneurysm A along its full length, thus excluding blood from the aneurysm by preventing blood flow between outer surface 16 and inner surface 17.

Catheter 14 includes an elongate catheter body 15, an infusion port 24 for introducing an inflation medium into inflation lumen 18 of expandable member 12, and a trigger 26 for disconnecting catheter 14 from expandable member 12. In various embodiments, the proximal end of catheter 14 may include additional ports 25 and/or any of a number of additional features as are generally known in the art.

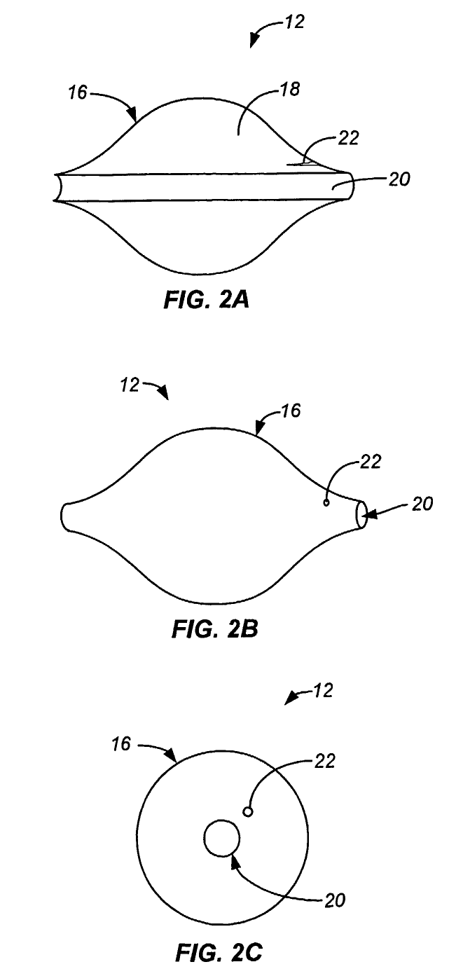

Expandable member 12 is shown more clearly in FIGS. 2A-2C. FIG. 2A shows expandable member 12 in cross section, FIG. 2B shows it in perspective view, and FIG. 2C shows expandable member 12 in end-on view. Generally, expandable member 12 may be constructed of any suitable elastomeric or other inflatable material or combination of materials, such as but not limited to polymers, such as PTFE. The size and shape of expandable member 12 will vary in different embodiments. In fact, in some embodiments, images of an aneurysm may be used to construct a three-dimensional model of the aneurysm, and the model may be used to form a custom expandable member 12 for treating that particular aneurysm. In other embodiments, a user may be provided multiple expandable members 12 of varying sizes and/or shapes and may choose a desired size and shape for a particular aneurysm. In one embodiment, for example, expandable member 12 may have a length of between about 10 mm and about 40 mm, an outer diameter of between about 6 mm and about 30 mm, and a blood flow lumen diameter of between about 3 mm and about 6 mm. The typical shape of expandable member 12 is fusiform.

The overall shape and size of expandable member 12 will cause it to contact inner surface 17 of the aneurysm A and to adhere or lock in place within the aneurysm A. In some embodiments, one or more additional features may be incorporated into expandable member 12 to help it to lock or adhere in place. For example, in some embodiments, expandable member 12 may have a coating on its outer surface 16 to promote adherence to inner surface 17 and/or tissue ingrowth into expandable member 12. Examples of such outer coatings include, but are not limited to, collagen, tissue adhesives and a polymeric matrix (e.g., as provided by Surmodics, Inc., Eden Prairie, Minn.). In other embodiments, multiple small hooks, anchors, barbs or the like may be placed on outer surface 16 to promote attachment to inner surface 17.

Expandable member 12 is releasably coupled to the distal end of delivery catheter 14, which is adapted to inflate and release expandable member 12. Any suitable mechanisms may be used to achieve inflation and release. In the embodiment shown, expandable member 12 includes self-sealing, one-way valve 22, which is removably coupled in fluid communication with catheter 14. An inflation medium, such as saline, is introduced into catheter 14 via inflation port 24, and travels through catheter 14 and one-way valve 22 to inflation lumen 18. When inflation is complete, catheter 14 releases expandable member 12, via a latch or other release/attachment mechanism controlled by trigger 26 or any alternative suitable means. When catheter 14 releases expandable member 14, it also disengages from valve 22, thus causing valve 22 to seal, holding the inflation medium within inflation lumen 18.

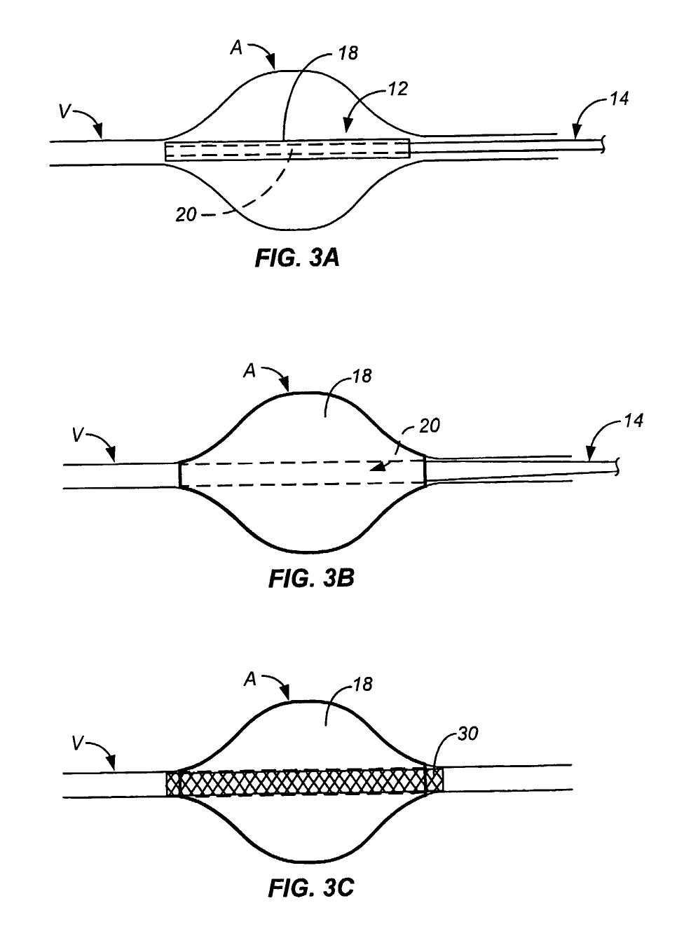

Referring now to FIGS. 3A-3C, a method for deploying expandable member 12 to treat a fusiform aneurysm A in a vessel V is illustrated. As shown in FIG. 3A, catheter 14 is advanced through the vessel V to position expandable member 12 across the aneurysm A. A number of different features on catheter 14 and/or expandable member 12 may be used to assist in and confirm correct positioning. For example, expandable member 12 may have one or more radiopaque markers or be made of one or more radiopaque materials. Catheter 14 may also include radiopaque markers or depth markers on its outer surface, showing how far catheter 14 has been advanced into the patient.

Once expandable member 12 is in a desired location relative to the aneurysm A, inflation medium, such as saline, is infused into inflation lumen 18 to cause expansion, as in FIG. 3B. Catheter 14 is then removed, leaving expandable member 12 in the aneurysm A, with blood being able to flow freely through blood flow lumen 20. In an additional, optional step, and referring now to FIG. 3C, one or more stent devices 30 or similar prostheses may be placed through blood flow lumen 20. Stent device 30 may help support blood flow lumen 20 and may also help anchor expandable member 12 within the aneurysm A. In some embodiments, stent 30 may extend through only a portion of blood flow lumen 20, while in others, it extends through the entire lumen 20 and protrudes from each end to contact the vessel wall, as shown in FIG. 3C. Such contact with vessel wall typically aids in the anchoring function.

During and/or after deployment of expandable member 12 in the aneurysm, proper placement and expansion of expandable member 12 may be confirmed via one or more imaging techniques. For example, if expandable member 12 includes radiopaque markers or material(s), or if radiopaque inflation fluid is used, various radiographic images may be acquired to help assess proper positioning and expansion. In some embodiments, one or more angiograms may be taken before, during and/or after expansion of expandable member 12, to confirm that no blood is flowing between outer surface 16 of fully expanded expandable member 12 and inner surface 17 of the aneurysm A.

Although the invention has been described above with specific reference to various embodiments and examples, various additions, modifications, deletions and alterations may be made to such embodiments without departing from the spirit or scope of the invention. Accordingly, it is intended that all reasonably foreseeable additions, deletions, alterations and modifications be included within the scope of the invention as defined in the following claims. All publications, patents, and patent applications cited herein are hereby incorporated by reference in their entirety for all purposes.

What is claimed is:

1. A device for treating a fusiform aneurysm, the device comprising:
an elongate catheter having a proximal end and a distal end; and
an expandable member detachably coupled with the elongate catheter at or near the distal end and adapted to assume a fusiform expanded shape such that an external surface of the expandable member will contact an inner wall of the aneurysm, thus preventing blood from passing between the external surface of the expandable member and the inner wall of the aneurysm, the expandable member comprising:
an inflation lumen to allow expansion of the expandable member; and
a blood flow lumen extending through the expandable member to allow blood to flow from a portion of a blood vessel proximal to the aneurysm, through the blood flow lumen, to a portion of the blood vessel distal to the aneurysm,
wherein the expandable member retains an expanded configuration and remains in place within the aneurysm when detached from the elongate catheter body.

2. A device as in claim 1, wherein the fusiform aneurysm is an intracranial aneurysm.

3. A device as in claim 1, further comprising at least one substance coupled with the external surface of the expandable member for adhering the external surface to the inner wall of the aneurysm.

4. A device as in claim 3, wherein the substance is selected from the group consisting of collagen, a tissue adhesive or a polymeric matrix.

5. A device as in claim 1, wherein the expandable member has a length of between 10 mm and 40 mm, an outer diameter of between 6 mm and 30 mm, and a blood flow lumen diameter of between 3 mm and 6 mm.

6. A device as in claim 1, further comprising at least one expandable stent adapted for balloon expansion within the blood flow lumen of the expandable member to support the blood flow lumen.

7. A device as in claim 1, further comprising at least one self-expanding stent adapted to expand within the blood flow lumen of the expandable member upon release from constraint to support the blood flow lumen.

8. A device as in claim 6, wherein the catheter is adapted to deploy at least one stent within the blood flow lumen of the expandable member.

9. A device as in claim 7, wherein the catheter is adapted to deploy at least one stent within the blood flow lumen of the expandable member.

10. A device as in claim 1, wherein the expandable member further comprises a self-sealing, one-way valve for detachably coupling the expandable member to the catheter, wherein the valve allows inflation medium to be introduced into the expandable member while preventing it from escaping, and wherein detaching the catheter from the expandable member seals the valve.

11. A device as in claim 10, wherein the valve detachably couples the expandable member to the catheter electrolytically.

12. A device as in claim 10, wherein the valve detachably couples the expandable member to the catheter hydrostatically.

13. A device as in claim 1, wherein the expandable member further comprises at least one anchoring member coupled with its outer surface, the anchoring member adapted to anchor the expandable member to the inner wall of the aneurysm.

14. A device as in claim 1, wherein the expandable member further comprises at least one radiopaque material or marker for facilitating visualization of the device.

15. A method for treating a fusiform aneurysm, the method comprising:
expanding an expandable member into a fusiform shape within the aneurysm to cause an external surface of the expandable member to contact an inner wall of the aneurysm to prevent blood from passing between the external surface of the expandable member and the inner wall of the aneurysm, the expandable member comprising:
an inflation lumen to allow expansion of the expandable member; and
a blood flow lumen extending through the expandable member to allow blood to flow from a portion of a blood vessel proximal to the aneurysm, through the blood flow lumen, to a portion of the blood vessel distal to the aneurysm; and
leaving the expanded expandable member within the aneurysm to continue preventing blood from passing between the external surface of the expandable member and the inner wall of the aneurysm and allowing blood to flow through the blood flow lumen.

16. A method as in claim 15, wherein the expandable member is expanded via an elongate catheter device detachably coupled with the expandable member and in fluid communication with the inflation lumen, and wherein leaving the expandable member within the aneurysm comprises detaching the catheter from the expandable member.

17. A method as in claim 16, wherein the expandable member is inflated with saline.

18. A method as in claim 16, wherein detaching the catheter from the !expandable member automatically seals a one-way inflation valve on the expandable member.

19. A method as in claim 15, further comprising placing at least one stent within the blood flow lumen to support the blood flow lumen.

20. A method as in claim 19, wherein the expandable member is expanded and the stent is placed using one catheter device.

21. A method as in claim 15, further comprising performing an angiogram of the aneurysm during the expanding step to determine whether blood is flowing between the external surface of the expandable member and the inner wall of the aneurysm.

22. A method as in claim 21, further comprising performing an angiogram of the aneurysm after the expanding step to confirm the absence of blood between the external surface of the expandable member and the inner wall of the aneurysm.

23. A method for making an expandable device to treat a fusiform aneurysm, the method comprising:
acquiring at least one image of the fusiform aneurysm;
preparing at least one three-dimensional model of the fusiform aneurysm, based on the at least one image; and
forming the expandable device to have a shape, when expanded, approximating that of the three-dimensional model.

24. A method as in claim 23, wherein the model comprises a mold, and wherein forming the expandable device comprises disposing an elastomeric material over the mold.

25. A method as in claim 23, wherein the expandable device comprises an inflatable balloon having at least one self-sealing, one-way valve.
